(19) United States
(12) Patent Application Publication
(10) Pub. No.: US 2025/0353831 A1
(43) Pub. Date: Nov. 20, 2025

(54) NOVEL HETEROBICYCLIC COMPOUND FOR INHIBITING YAP-TEAD INTERACTION AND PHARMACEUTICAL COMPOSITION COMPRISING SAME

(71) Applicant: HANMI PHARM. CO., LTD., Hwaseong-si, Gyeonggi-do (KR)

(72) Inventors: Min Jeong KIM, Hwaseong-si (KR); Ji Sook KIM, Hwaseong-si (KR); Hee Sun MOON, Hwaseong-si (KR); Kwee Hyun SUH, Hwaseong-si (KR); Young Gil AHN, Hwaseong-si (KR); Ji Hee YOON, Hwaseong-si (KR); Seung Hyun JUNG, Hwaseong-si (KR); Seon Yeong HAN, Hwaseong-si (KR)

(21) Appl. No.: 19/120,165

(22) PCT Filed: Oct. 12, 2023
(86) PCT No.: PCT/KR2023/015759

§ 371 Date: Apr. 10, 2025

(30) Foreign Application Priority Data
Oct. 13, 2022 (KR) 10-2022-0131763

(51) Int. Cl. C07D 403/04; A61K 31/404; A61K 31/4178; A61K 31/437; A61P 35/00; C07D 209/14; C07D 405/04; C07D 471/04

(52) U.S. Cl. CPC C07D 403/04; A61K 31/404; A61K 31/4178; A61K 31/437; A61P 35/00; C07D 209/14; C07D 405/04; C07D 471/04

(57) ABSTRACT

Provided are a compound, selected from compounds of Formula 1, enantiomers, diastereomers, solvates and hydrates thereof, and pharmaceutically acceptable salts thereof, a method of manufacturing the same, and use thereof.

### TECHNICAL FIELD

The present disclosure relates to a pharmaceutical composition including a hetero-bicyclic compound that inhibits Yes associated protein (YAP)-transcriptional enhancer associate domain (TEAD) binding. The compound of the present disclosure may directly inhibit YAP-TEAD binding in the Hippo pathway that plays a key role in a cancer development process.

### BACKGROUND ART

The Hippo signaling cascade is an important pathway of cancer biogenesis and tumor maintenance. YAP and TAZ are transcriptional co-activators of the Hippo pathway network and regulate cell proliferation, migration, and apoptosis. Inactivation of the Hippo signaling pathway promotes YAP/TAZ translocation to nuclei, wherein YAP/TAZ interact with transcriptional enhancer associate domain (TEAD) transcription factors, coactivate expression of target genes, and promote cell proliferation. Target genes closely associated with oncogenesis, such as connective tissue growth factor (CTGF) and Cyr61, AXL receptor tyrosine kinase, and MYC are regulated by TEAD. Also, TEAD was found to be overexpressed in breast cancer stem cells and breast cancer, ovarian cancer, germ cell tumor, renal cell carcinoma, medulloblastoma, and gastric cancer. Hyperactivation of YAP and TAZ and/or mutations in one or more members of the Hippo pathway network are related to numerous cancers. Furthermore, recent studies have reported that resistance to EGFR tyrosine kinase inhibitors, such as Tarceva (erlotinib), Iressa (gefitinib) or Tagrisso (osimertinib) is related to YAP over-expression or YAP amplification together with epithelial-mesenchymal transition (EMT) phenotype changes.

The present inventors have developed a novel heterobicyclic compound for inhibiting YAP-TEAD interaction, thereby completing the present disclosure.

### PRIOR ART DOCUMENTS

### Patent Documents

(Patent Document 1) International Publication No. WO2019040380
(Patent Document 2) International Publication No. WO2020243415

### Non-patent Documents

(Non-patent Document 1) Semin. Cancer Biol. 2022, 85, 33
(Non-patent Document 2) Nat. Rev. Drug Discov. 2014, 13(1), 63
(Non-patent Document 3) Cancer Res.2011, 71(3), 873
(Non-patent Document 4) J. Cell Mol. Med.2017, 21(11), 2663
(Non-patent Document 5) Cancer Cell2020, 37, 104
(Non-patent Document 6) Cells2021, 10, 2715
(Non-patent Document 7) Genes Cancer2017, 8(3-4), 497

### DISCLOSURE OF INVENTION

### Technical Problem

An object of the present disclosure is to provide a novel heterobicyclic compound having excellent inhibitory activity against YAP-TEAD binding in the Hippo pathway that plays a key role in a cancer development process.

Another object of the present disclosure is to provide a pharmaceutical composition for treating or preventing a related disease caused by dysregulation of the Hippo signaling pathway, specifically, TEAD activation, the pharmaceutical composition including the compound as an active ingredient.

Other objects and advantages of the present application will become more apparent by the following detailed description in conjunction with the appended claims and drawings. Contents not described in the present specification will be omitted because they can be sufficiently recognized and inferred by a person skilled in the art of the present application or in a similar technical field.

### Solution to Problem

According to an embodiment of the present disclosure, provided is a compound selected from compounds of Formula 1 below, enantiomers, diastereomers, solvates and hydrates thereof, and pharmaceutically acceptable salts thereof.

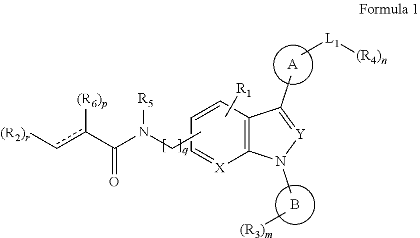

According to an embodiment of the present disclosure, provided is a pharmaceutical composition for treating or preventing a related disease caused by dysregulation of the Hippo signaling pathway, specifically, TEAD activation, the pharmaceutical composition including, as an active ingredient, the compound selected from compounds of Formula 1, enantiomers, diastereomers, solvates and hydrates thereof, and pharmaceutically acceptable salts thereof.

### Advantageous Effects of Invention

A novel heterobicyclic compound having a structure of Formula 1 according to the present disclosure may have effects on diseases associated with the Hippo pathway, which plays a key role in a cancer development process, due to excellent inhibitory activity against YAP-TEAD binding, and thus may be used effectively as a therapeutic agent.

### MODE FOR THE INVENTION

Hereinafter, the present disclosure will be described in detail.

Unless otherwise defined, all technical terms used herein have the same meaning as commonly understood by one of ordinary skill in the art to which the present disclosure belongs. Although preferred methods or samples are described in the specifications, those similar or equivalent thereto may also be regarded within the scope of the present disclosure.

According to an embodiment of the present disclosure, provided is a compound selected from compounds of Formula 1 below, enantiomers, diastereomers, solvates and hydrates thereof, and pharmaceutically acceptable salts thereof.

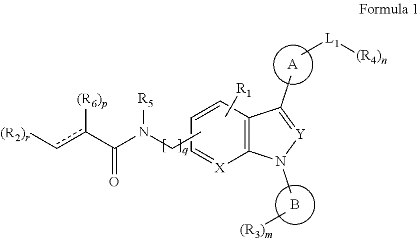

In Formula 1,
R₁ may be hydrogen, halogen, C₁₋₆ alkyl, halo C₁₋₆ alkyl, C₁₋₆ alkoxy, or cyano;
R₂ may each independently be hydrogen, halogen, C₁₋₆ alkyl, halo C1-₁₋₆ alkyl, or substituted or unsubstituted —(CH₂)ₐ—N(Q₁)(Q₂),
wherein Q₁ and Q₂ may each independently be hydrogen or C₁₋₄ alkyl;
\textcircled{A} may be carbocyclyl or heterocyclyl;
\textcircled{B} may be C₆₋₁₀ aryl or C₄₋₁₀ heteroaryl;
L₁ may be absent, a bond, C₁₋₃ alkyl, or halo C₁₋₃ alkyl;
R₃ and R₄ may each independently be hydrogen, halogen, cyano, amino, C₁₋₆ alkyl, halo C₁₋₆ alkyl, C₁₋₆ alkoxy, halo C₁₋₆ alkoxy, C₁₋₆ alkoxyalkyl, substituted or unsubstituted C₃₋₆ carbocyclyl, substituted or unsubstituted C₆₋₁₀ aryl, substituted or unsubstituted C₂₋₆ heterocyclyl, or substituted or unsubstituted C₄₋₁₀ heteroaryl;
R₅ may be hydrogen, C₁₋₆ alkyl, or halo C₁₋₆ alkyl;
R₆ may each independently be hydrogen, halogen, C₁₋₆ alkyl, or halo C₁₋₆ alkyl;
X and Y may each independently be —C— or —N—; and
a, m, n, q, r, and p may each independently be an integer from 0 to 3.

As used herein, the term “halogen” may be F, Cl, Br, or I.

As used herein, unless otherwise stated, the term “alkyl” refers to a straight- or branched-chain hydrocarbon residue that may be substituted or unsubstituted. The alkyl group may be, for example, methyl, ethyl, propyl, butyl, pentyl, isopropyl, isobutyl, or t-butyl, but is not limited thereto.

As used herein, unless otherwise stated, the term “alkenyl” refers to an alkyl group including at least one double bond that may be substituted or unsubstituted. The alkenyl group may be, for example, prop-1-ene, but-1-ene, but-2-ene, 3-methylbut-1-ene, or pent-1-ene, but is not limited thereto.

As used herein, unless otherwise stated, the term “cycloalkyl” refers to a saturated monocyclic and polycyclic hydrocarbon ring generally including a specified number of carbon atoms having a ring that may be substituted or unsubstituted. The cycloalkyl group may be, for example, cyclopropyl, cyclobutyl, cyclopentyl, cyclohexyl, or cycloheptyl, but is not limited thereto.

As used herein, unless otherwise stated, the term “heterocycloalkyl” refers to a monocyclic alkyl including at least one hetero atom selected from N, O, and S that may be substituted or unsubstituted. The heterocycloalkyl group may be, for example, piperidinyl, piperazinyl, morpholinyl, pyrrolidinyl, thiomorpholinyl, imidazolidinyl, tetrahydrofuranyl, or similar groups thereof, but is not limited thereto.

As used herein, unless otherwise stated, the term “haloalkyl” refers to include monohaloalkyl and polyhaloalkyl, which may be substituted or unsubstituted. The terms “halogen” and “alkyl” are as described above.

As used herein, unless otherwise stated, the term “alkoxy” refers to a straight- or branched-chain hydrocarbon residue linked via oxygen that may be substituted or unsubstituted. The alkoxy group may be, for example, methoxy, ethoxy, propoxy, and butoxy, or isopropoxy, isobutoxy, or t-butoxy, but is not limited thereto.

As used herein, the term “alkoxyalkyl” refers to alkyl group, one or more hydrogen atoms thereof are substituted with one or more alkoxy groups. The alkoxyalkyl may be, for example, methoxymethyl, ethoxymethyl, methoxyethyl, ethoxyethyl, methoxypropyl, ethoxypropyl, and isopropoxymethyl, but is not limited thereto.

As used herein, unless otherwise stated, the term “aryl” refers to an aromatic group that may be substituted or unsubstituted, and may include, for example, C₃-C₁₀ aryl, C₃-C₈ aryl, or C₃-C₆ aryl, in which double bonds are alternately placed (resonate) between adjacent carbon atoms or suitable heteroatoms. For example, the aryl may be phenyl, biphenyl, naphthyl, toluyl, or naphthalenyl, but is not limited thereto.

As used herein, unless otherwise stated, the term “heteroaryl” refers to a monocyclic, bicyclic, or multicyclic aromatic group including at least one heteroatom selected form N, O, and S that may be substituted or unsubstituted. For example, the monocyclic heteroaryl may be pyridinyl, imidazolyl, thiazolyl, oxazolyl, thiophenyl, furanyl, pyrrolyl, isoxazolyl, pyrazolyl, triazolyl, thiadiazolyl, tetrazolyl, oxadiazolyl, pyridazinyl, pyrimidinyl, or pyrazinyl, but is not limited thereto. For example, the bicyclic heteroaryl may be indolyl, benzothiophenyl, benzofuranyl, benzimidazolyl, benzoxazolyl, benzisoxazolyl, benzthiazolyl, benzthiadiazolyl, benztriazolyl, quinolinyl, isoquinolinyl, furanyl, or furopyridinyl, but is not limited thereto.

As used herein, unless otherwise stated, the term “carbocyclyl” refers to a substituent including a carbon ring atom having a saturated carbocyclyl (e.g., “cycloalkyl”), a partially saturated carbocyclyl (e.g., “cycloalkenyl”), or a completely unsaturated carbocyclyl (e.g., “aryl”) structure. The carbocyclyl may have a monocyclic or polycyclic ring structure. As used herein, the carbocyclyl includes, for example, 3 to 14, or, for example, 3 to 8, carbon ring atoms, and may be saturated, unsaturated, or aromatized. In this regard, the ring atoms are atoms joined together to form a ring or rings of the carbocyclyl substituent. For example, the saturated carbocyclyl group may be cyclopropyl, cyclopentyl, or cyclohexyl, but is not limited thereto. For example, the unsaturated carbocyclyl group may include 3 or less double bonds. For example, the aromatic carbocyclyl group may be phenyl. Also, the term “carbocyclyl” may include a fused combination of carbocyclyl groups, such as naphthyl, phenanthryl, indanyl, and indenyl, but is not limited thereto.

As used herein, unless otherwise stated, the term “heterocyclyl” refers to a substituent including a carbon ring with at least one heteroatom and having a saturated heterocyclyl (e.g., “heterocycloalkyl”), partially saturated heterocyclyl (e.g., “heterocycloalkenyl”), or completely unsaturated heterocyclyl (e.g., “heteroaryl”) structure. The heterocyclyl may have a monocyclic or polycyclic ring structure. As used herein, the heterocyclyl includes, for example, a total of 3 to 14, or for example, a total of 3 to 8 ring atoms, and may be saturated, unsaturated, or aromatized. In this regard, the ring atoms are atoms joined together to form a ring or rings of the heterocyclyl substituent. For example, at least one of the ring atoms is nitrogen, oxygen, or sulfur, and the other ring atoms are each independently selected from carbon, nitrogen, oxygen, and sulfur. For example, the ring atom of the heterocyclyl may include 4 or less heteroatoms such as N, O, and S, for example, a total of 3 to 14, or for example, a total of 5 to 7, ring atoms, and may be saturated, unsaturated, or aromatized. For example, the heterocyclyl may be furanyl, thiophenyl, pyrrolyl, pyrrolinyl, pyrrolidinyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, tetrazolyl, thiadiazolyl, pyranyl, pyridinyl, piperidinyl, dioxanyl, morpholino, dithianyl, thiomorpholino, pyridazinyl, pyrimidinyl, pyrazinyl, piperazinyl, sulfolanyl, triazinyl, azepinyl, oxazepinyl, thiazepinyl, diazepinyl, or thiazolinyl, but is not limited thereto. In addition, the term “heterocyclyl” may include a fused heterocyclyl group, for example, benzimidazolinyl, benzoxazolyl, imidazopyridinyl, benzoxazinyl, benzothiazinyl, oxazolopyridinyl, quinolinyl, quinazolinyl, quinoxazolinyl, dihydroquinazolinyl, benzothiazolyl, phthalimido, benzofuranyl, benzodiazepinyl, indolyl, or isoindolyl, but is not limited thereto. The “heterocyclyl” may be a carbon linker or a heteroatom linker. For example, in the heteroatom linker, an N-linked heterocyclyl includes

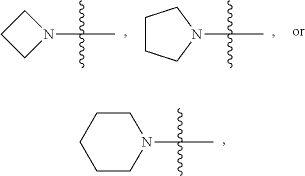 [SMILES: *N1CCC1 ; *N1CCCC1 ; *N1CCCCC1]

but is not limited thereto.

As used herein, unless otherwise stated, the term “fused heteroaryl” refers to a substituted or unsubstituted ring system in which a heteroaryl group is linked with another aryl, heteroaryl, or heterocycloalkyl group in a fused manner. For example, the fused heteroaryl may constitute a 5+5-membered, 5+6-membered, 5+7-membered, 6+6-membered, or 6+7-membered fused ring system. Also, the fused heteroaryl may be, for example,

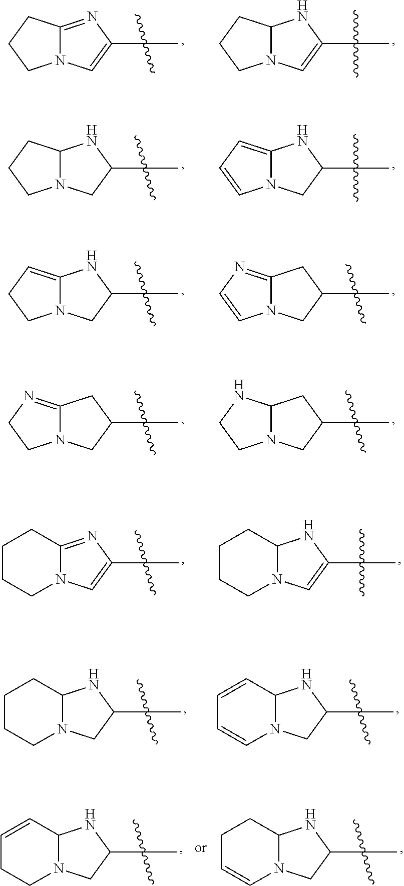

but is not limited thereto.

The substituent used herein may be one selected from, for example, cyano, amino, hydroxyl, C₁₋₆ alkyl, halo C₁₋₆ alkyl, C₁₋₆ alkoxy, and halo C₁₋₆ alkoxy, but is not limited thereto.

For example, the substituted C₃₋₆ cycloalkyl, C₆₋₁₀ aryl, or C₂₋₆ heterocycloalkyl may be alkyl groups, one or more hydrogen atoms of which are substituted with one substituent selected from halogen, cyano, amino, hydroxyl, C₁₋₆ alkyl, halo C₁₋₆ alkyl, C₁₋₆ alkoxy, and halo C₁₋₆ alkoxy, but are not limited thereto.

As used herein, the term “stereoisomer” may refer to the compound of the present disclosure or a salt thereof that has the same chemical or molecular formula but is optically or sterically different, and may include enantiomers or diastereomers.

As used herein, the term “enantiomer” refers to a pair of stereoisomers of a compound that are non-superimposable mirror images of each other.

As used herein, the term “diastereomer” refers to stereoisomers with two or more centers of chirality and whose molecules are not mirror images of one another.

The compound of the present disclosure may include asymmetric or chiral centers and therefore may exist in different stereoisomeric forms. It is intended that all stereoisomeric forms of the compounds of the present disclosure such as diastereomers, enantiomers, and racemic mixtures form part of the present disclosure. An equimolar mixture (50:50) of two enantiomers is called racemic mixture or racemate.

As used herein, the term “solvate” refers to the compound of the present disclosure or a salt thereof containing a stoichiometric or non-stoichiometric amount of a solvent bound by non-covalent intermolecular forces. As solvents suitable therefor may be volatile, non-toxic, and/or solvents suitable for administration to humans. The “solvate” may include the compound and a molecular complex including one or more pharmaceutically acceptable solvent molecules, e.g., ethanol.

As used herein, the term “hydrate” refers to a complex wherein the solvent molecules are complexes.

As used herein, the term “pharmaceutically acceptable salt” refers to a pharma- ceutically acceptable organic or inorganic salt and may be prepared by any suitable method available to those skilled in the art. For example, if the compound of the present disclosure is a base, a desired pharmaceutically acceptable salt may be prepared by any suitable method available to those skilled in the art, for example, by treating the free base with an inorganic acid, an organic acid, or the like.

In an embodiment, \textcircled{A} may be C₃₋₆ cycloalkyl, C₆₋₁₀ aryl, C₂₋₆ heterocycloalkyl, C₁₋₁₀ heteroaryl, or C₆₋₁₄ fused heteroaryl,
wherein the C₂₋₆ heterocycloalkyl, C₁₋₁₀ heteroaryl, or C₆₋₁₄ fused heteroaryl may each independently include 1 to 4 heteroatoms selected from N, O, and S.

In an embodiment, \textcircled{A} may be a phenyl group, a pyridinyl group, a pyrazinyl group, a pyrazolyl group, an imidazolyl group, a thiophenyl group, a furanyl group, or an oxazole group.

In an embodiment, \textcircled{B} may be C₆₋₁₀ aryl.

In an embodiment, \textcircled{B} may be a phenyl group or a pyridinyl group.

In an embodiment, R₁ may be hydrogen, halogen, or cyano.

In an embodiment, R₂ may each independently be hydrogen, halogen, or —CH₂—NH₂, —CH₂—NHCH₃, —CH₂—N(CH₃)₂.

In an embodiment, R₂ may each independently be hydrogen or halogen.

In an embodiment, R₅ may be hydrogen.

In an embodiment, R₆ may each independently be hydrogen or halogen.

In an embodiment, L₁ may be a bond; and
R₃ and R₄ may each independently be hydrogen, halogen, cyano, C₁₋₆ alkyl, halo C₁₋₆ alkyl, C₁₋₆ alkoxy, C₁₋₆ alkoxyalkyl, substituted or unsubstituted C₃₋₆ cycloalkyl, substituted or unsubstituted C₆₋₁₀ aryl, or substituted or unsubstituted C₂₋₆ heterocycloalkyl.

In an embodiment,
\textcircled{A} may be a phenyl group, an imidazolyl group, or a furanyl group;
\textcircled{B} may be a phenyl group;
L₁ may be a bond;
R₃ may be halo C₁₋₆ alkyl;
R₄ may be hydrogen, halogen, C₁₋₆ alkyl, halo C₁₋₆ alkyl, C₁₋₆ alkoxy, C₁₋₆ alkoxyalkyl, or C₃₋₆ cycloalkyl;
R₁ may be hydrogen or halogen;
R₂ and R₅ may be hydrogen;
R₆ may each independently be hydrogen or halogen;
X and Y may each independently be —C— or —N—; and

m, n, q, r, and p may each independently be an integer from 0 to 2.

In an embodiment,
\textcircled{A} may be

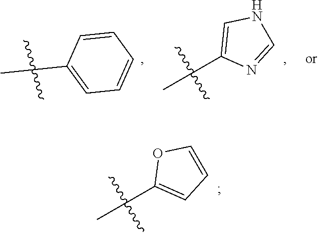 [SMILES: *c1ccccc1 ; *c1c[nH]cn1 ; *c1ccco1]

\textcircled{B} may be

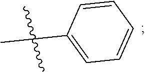 [SMILES: *c1ccccc1]

L₁ may be a bond;
R₃ may be halo C₁₋₆ alkyl;
R₄ may be hydrogen, halogen, C₁₋₆ alkyl, C₁₋₆ alkoxy, or C₃₋₆ cycloalkyl;
R₁ may be hydrogen or halogen;
R₂ and R₅ may each independently be hydrogen;
R₆ may each independently be hydrogen or halogen;
X may be carbon or nitrogen;
Y may be carbon; and
m, n, q, r, and p may each independently be an integer from 0 to 2.

In an embodiment, the compound may be selected from the following compounds, enantiomers, diastereomers, solvates and hydrates thereof, and pharmaceutically acceptable salts thereof.
1) N-(3-(1-methyl-1H-imidazol-4-yl)-1-(4-(trifluoromethyl)phenyl)-1H- indol-5-yl)acrylamide;
2) N-(3-(1-cyclopropyl-1H-imidazol-4-yl)-1-(4-(trifluoromethyl)phenyl)-1H- indol-5-yl)acrylamide;
3) N-(3-(furan-2-yl)-1-(4-(trifluoromethyl)phenyl)-1H-indol-5-yl) acrylamide;
4) N-(3-(1-cyclobutyl-1H-imidazol-4-yl)-1-(4-(trifluoromethyl)phenyl)-1H- indol-5-yl)acrylamide;
5) N-(3-(2-fluorophenyl)-1-(4-(trifluoromethyl)phenyl)-1H-indol-5-yl) acrylamide;
6) 2-fluoro-N-(3-(1-methyl-1H-imidazol-4-yl)-1-(4-(trifluoromethyl)phenyl)-1H- indol-5-yl)acrylamide;
7) N-(3-(1-(2-methoxyethyl)-1H-imidazol-4-yl)-1-(4-(trifluoromethyl)phenyl)-1H- indol-5-yl)acrylamide;
8) N-(3-(1-cyclopropyl-1H-imidazol-4-yl)-1-(4-(trifluoromethyl)phenyl)-1H- pyrrolo[2,3-b]pyridin-5-yl)acrylamide;
9) N-(3-(1-methyl-1H-imidazol-4-yl)-1-(4-(trifluoromethyl)phenyl)-1H-pyrrolo[2,3-b]pyridin-5-yl)acrylamide; and
10) N-(6-chloro-3-(1-methyl-1H-imidazol-4-yl)-1-(4-(trifluoromethyl)phenyl)-1H- indol-5-yl)acrylamide.

According to an embodiment of the present disclosure, provided is a pharmaceutical composition for treating or preventing a related disease caused by dysregulation of the Hippo signaling pathway, specifically, TEAD activation, the pharmaceutical composition including, as an active ingredient, the compound selected from compounds of Formula 1, enantiomers, diastereomers, solvates and hydrates thereof, and pharmaceutically acceptable salts thereof.

In an embodiment, the composition may exhibit inhibitory activity against binding of Yes associated protein (YAP) to transcriptional enhancer associate domain (TEAD).

In an embodiment, the composition may be used to treat cancers or tumors curable by inhibitory activity against YAP-TEAD binding.

In an embodiment, the pharmaceutical composition may include a compound selected from the compounds of Formula 1, enantiomers, diastereomers, solvates and hydrates thereof, and pharmaceutically acceptable salts thereof in a therapeutically effective amount.

As used herein, the term “therapeutically effective amount” refers to an amount of the compound of the present disclosure required to treat or prevent a certain disease, condition, or disorder, to alleviate, beneficially change, or remove one or more symptoms of a certain disease, condition, or disorder, or to prevent or delay onset of one or more symptoms of a certain disease, condition, or disorder.

An effective dose of the pharmaceutical composition may be determined and prescribed by a physician skilled in the art. For example, the pharmaceutical composition may include the compound in an amount of 0.0001 mg to 10 g, without being limited thereto.

In an embodiment, the pharmaceutical composition may further include a pharmaceutically acceptable additive in addition to the active ingredient. The additive may be, for example, a diluent, a disintegrant, a binder, a lubricant, a surfactant, a suspending agent, or an emulsifier, but is not limited thereto.

The pharmaceutical composition of the present disclosure may be formulated according to any known method and may be prepared into various dosage forms for oral administration such as tablets, pills, powders, capsules, syrups, emulsions, and microemulsions or for parenteral administration such as intramuscular, intravenous, or subcutaneous administration.

According to another embodiment of the present disclosure, provided is a therapeutic method of administering the pharmaceutical composition to a subject having a related disease caused by dysregulation of the Hippo signaling pathway, specifically, TEAD activation, the pharmaceutical composition including, as an active ingredient, the compound selected from compounds of Formula 1, enantiomers, diastereomers, solvates and hydrates thereof, and pharmaceutically acceptable salts thereof.

As used herein, the term “treating” or “treatment” refers to inhibiting a disease, e.g., inhibiting a disease, condition, or disorder in an individual experiencing or having pathology or symptomatology of the disease, condition, or disorder, e.g., preventing or reversing further development of pathology and/or symptomatology or alleviating the disease, e.g., reducing severity of the disease.

As used herein, the term “preventing” or “prevention” refers to preventing a disease, e.g., preventing a disease, condition, or disorder in an individual who may be predisposed to the disease, condition or disorder but does not yet experience or display the pathology or symptomatology of the disease.

As used herein, the term “subject” or “individual” may be a vertebrate such as mammal, fish, bird, reptile, or amphibian. For example, the subject may be humans, non-human primates, horses, pigs, rabbits, dogs, sheep, goats, cows, cats, guinea pigs, or rodents.

As used herein, the term “administering” and “administration” refers to a method of providing the composition to a subject.

Administration dosage, number, or method of the compound or the pharmaceutical composition according to an embodiment may vary according to a subject to be treated, severity of a disease or condition, administration rate, and judgement of a prescribing physician. For example, a dosage for a person weighing 70 kg may generally be from 0.0001 mg to 10 g, for example, from 1 mg to 1 g, per day. The number of administration may be from once to multiple times, for example, once to four times, or by an on/off schedule, and the administration method may be performed via an oral or parenteral route. For example, the compound or the pharmaceutical composition according to an embodiment may be administered via an oral or parenteral route in an amount of 0.1 to 100 mg/kg (body weight).

Physicians may start with a dosage of the compound or the pharmaceutical composition of the present disclosure administered to a subject at a lower level than that required to obtain a target therapeutic effect and gradually increase the dosage until a desired effect is achieved.

According to another embodiment of the present disclosure, provided is a kit including a compound selected from the compounds of Formula 1, enantiomers, diastereomers, solvates and hydrates thereof, and pharmaceutically acceptable salts thereof as an active ingredient.

In an embodiment, the therapeutic agent may be a drug for treatment of a related disease caused by dysregulation of the Hippo signaling pathway, specifically, TEAD activation, e.g., a drug for treatment of cancer. For example, the therapeutic agent may be a chemotherapy drug for treatment of cancer.

In an embodiment, the compound, composition, and kit of the present disclosure may be administered alone or in combination with at least one other therapeutic agent simultaneously, separately, or sequentially.

In the context of the present disclosure, singular terms may include plural forms and vice versa unless otherwise stated.

Throughout the specification, numerical values are considered to include the meaning of “about” even if not specified. As used herein, the term “about” refers to a variation of a certain value or range within 5%, preferably, 1% to 2%.

As used herein, the term “to” refers to a range including numerical values provided before and after the term “to” as a lower limit and an upper limit thereof, respectively.

As used herein, the terms “have”, “may have”, “include”, or “may include” indicate the presence of corresponding features (e.g., numerical values or components such as ingredients) and do not exclude the existence of additional features.

The contents of all publications disclosed herein are incorporated by reference in their entirety.

Hereinafter, a method of manufacturing the compound of Formula 1 will be described in detail.

The compound of Formula 1 according to the present disclosure may be manufactured by a synthesis method represented by Reaction Scheme 1.

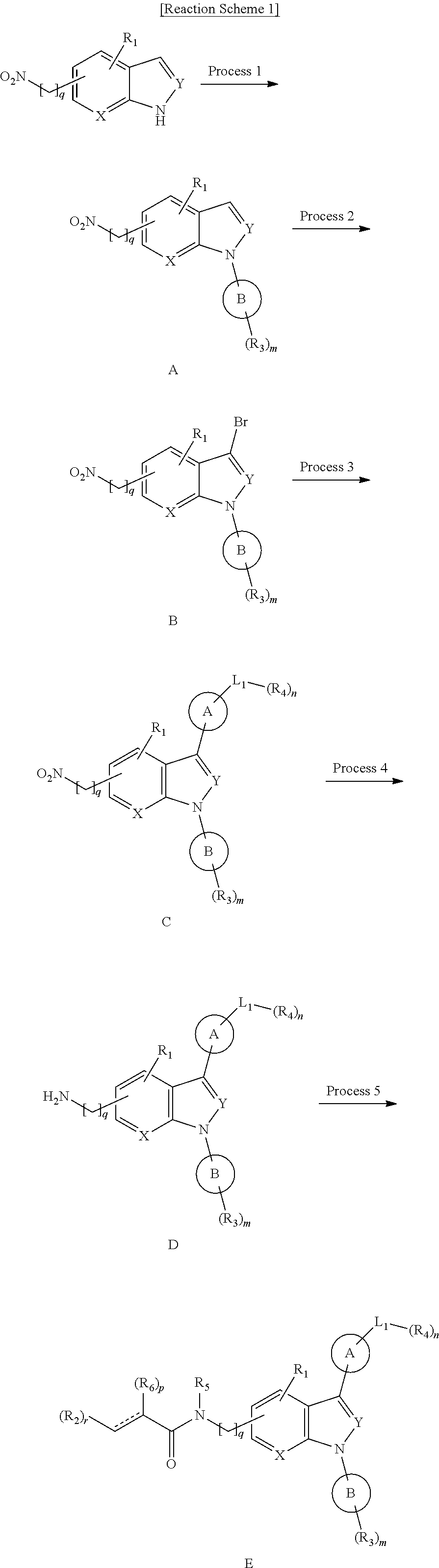

### Process-1

5-nitroindole (1 eq, standard equivalent 61.67 mmol) was dissolved in dimethyl- formamide, and a halogen-substituted B derivative (1.2 eq) and potassium carbonate (2 eq) were added thereto, and then copper (I) iodide (1 eq) was added thereto. The reaction solution was stirred at 120° C. overnight. Upon completion of the reaction, the reaction solution was cooled to room temperature, and water was added thereto, followed by extraction three times with ethylacetate. An organic layer was dried with anhydrous sodium sulfate and filtered under reduced pressure, and the filtered organic layer was concentrated under a reduced pressure. Residues obtained therefrom were purified by MPLC to obtain target Compound A.

### Process-2

Compound A (1 eq, standard equivalent) obtained in [Process-1] was dissolved in dichloromethane, and N-bromosuccinimide (1 eq) was slowly added thereto. The reaction solution was stirred at room temperature for 2 hours. After confirming completion of the reaction, water was added thereto, followed by extraction using dichloromethane. An organic layer was dried with anhydrous sodium sulfate and filtered under reduced pressure. After the filtered organic layer was concentrated under reduced pressure, residues obtained therefrom were purified by MPLC to obtain target Compound B.

### Process-3

Compound B (1 eq, standard equivalent) obtained in [Process-2] was subjected to Stille cross-coupling reaction or Suzuki coupling reaction with corresponding A-stannane derivative and A-borane derivative (2 eq). Upon completion of the reaction, the organic layer was washed with water, dried with anhydrous sodium sulfate, and filtered under reduced pressure, and then the filtered organic layer was concentrated under reduced pressure. Residues obtained therefrom were purified by MPLC to obtain target Compound C.

### Process-4

Compound C (1 eq, standard equivalent) obtained in [Process-3] was dissolved in ethanol and Pd/C (0.2 wt %) was added thereto. The reaction solution was stirred under a hydrogen gas for 16 hours. After confirming completion of the reaction, the reaction solution was filtered through CELITE and washed with methanol. The filtered organic layer was concentrated under reduced pressure to obtain target Compound D.

### Process-5

Compound D (1 eq, standard equivalent) obtained in [Process-4] was dissolved in a tetrahydrofuran: water mixture (3:1, v/v), and sodium bicarbonate (1.5 eq) was added thereto. The reaction solution was cooled to a temperature of 0 to 5° C. and stirred while slowing adding acryloyl chloride (1.05 eq) dropwise thereto. Upon completion of the dropwise addition, the reaction solution was stirred at 0 to 5° C. for 1 hour. After confirming completion of the reaction, water was added thereto, followed by extraction with ethylacetate. An organic layer was dried with anhydrous sodium sulfate and filtered under reduced pressure. After the filtered organic layer was concentrated under reduced pressure, residues obtained therefrom were purified by MPLC target Compound E.

In Reaction Scheme 1, R₁, R₂, R₃, R₄, R₅, R₆, X, Y, n, m, q, r, p, L₁, \textcircled{A}, and \textcircled{B} are as defined in Formula 1, but are not limited thereto, and changes may be made within the range understandable by those skilled in the art.

The compound of Formula 1 according to an embodiment of the present disclosure may be manufacture according to the method shown in Reaction Scheme 1 described above, but is not limited thereto. Those skilled in the field of organic compounds may be appropriately adjust reaction pathways, reaction conditions, reaction amounts, and the like.

Hereinafter, the present disclosure will be described in more detail with reference to the following examples and experimental examples. However, the following examples and experimental examples are merely presented to exemplify the present disclosure, and the scope of the present disclosure is not limited thereto.

### Example 1: N-(3-(1-methyl-1H-imidazol-4-yl)-1-(4-(trifluoromethyl)phenyl)-1H-indol-5-yl)acrylamide

[Process-1] Preparation of 5-nitro-1-(4-(trifluoromethyl)phenyl)-1H-indole

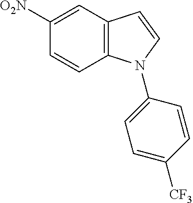 [SMILES: O=[N+]([O-])c1ccc2c(ccn2-c2ccc(C(F)(F)F)cc2)c1]

5-nitro-1H-indole (10.0 g, 61.67 mmol) was dissolved in 80 mL of dimethyl-formamide, and 4-bromobenzotrifluoride (10.6 mL, 74.00 mmol) and potassium carbonate (17.0 g, 123.34 mmol) were added thereto, and then copper (I) iodide (11.7 g, 61.67 mmol) was added thereto. The reaction solution was stirred at 120° C. overnight. Upon completion of the reaction, the reaction solution was cooled to room temperature, and 200 mL of water was added thereto, followed by extraction three times with ethylacetate. An organic layer was dried with anhydrous sodium sulfate and filtered under reduced pressure, and the filtered organic layer was concentrated under a reduced pressure. Residues obtained therefrom were purified by MPLC (ethylacetate:hexane=1:3(v/v)) to obtain 14 g of the title compound (yield: 74%).

¹H—NMR (300 MHz, CDCl₃): δ 8.69-8.68 (m, 1H), 8.20-8.16 (m, 1H), 7.89-7.86 (m, 2H), 7.68-7.65 (m, 2H), 7.60-7.51 (m, 2H), 6.94-6.93 (m, 1H).

[Process-2] Preparation of 3-bromo-5-nitro-1-(4-(trifluoromethyl)phenyl)-1H-indole

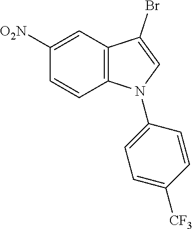 [SMILES: O=[N+]([O-])c1ccc2c(c1)c(Br)cn2-c1ccc(C(F)(F)F)cc1]

5-nitro-1-(4-(trifluoromethyl)phenyl)-1H-indole (14.0 g, 45.71 mmol) obtained in [Process-1] was dissolved in 140 mL of dichloromethane, and N-bromosuccinimide (8.3 g, 45.71 mmol) was slowly added thereto. The reaction solution was stirred at room temperature for 2 hours. Upon completion of the reaction, 50 mL of water was added thereto, followed by extraction three times with 50 mL of dichloromethane. An organic layer was dried with anhydrous sodium sulfate and filtered under reduced pressure. After the filtered organic layer was concentrated under reduced pressure, residues obtained therefrom were purified by MPLC (ethylacetate:hexane=1:3 (v/v)) to obtain 16.2 g of the title compound (yield: 92%).

¹H-NMR (300 MHz, CDCl₃): δ 8.62-8.61 (m, 1H), 8.22-8.18 (m, 1H), 7.88-7.85 (m, 2H), 7.64-7.61 (m, 2H), 7.57-7.54 (m, 2H).

[Process-3] Preparation of 3-(1-methyl-1H-imidazol-4-yl)-5-nitro-1-(4-(trifluoromethyl)phenyl)-1H-indole

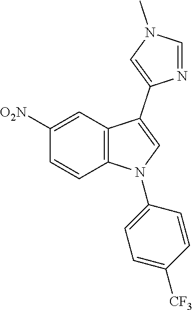 [SMILES: Cn1cnc(-c2cn(-c3ccc(C(F)(F)F)cc3)c3ccc([N+](=O)[O-])cc23)c1]

3-bromo-5-nitro-1-(4-(trifluoromethyl)phenyl)-1H-indole (13.0 g, 33.75 mmol) obtained in [Process-2] was dissolved in 260 mL of dimethylacetamide, and copper (I) chloride (689 mg, 6.75 mmol), cesium fluoride (15.8 g, 101.26 mmol), [1,1′-bis(diphenylphosphino)bisferrocenyl]palladium dichloride ([Pd(dppf)Cl₂], (5.2 g, 6.75 mmol)], and tributyl-(1-methylimidazol-4-yl)stannane (24.0 g, 64.80 mmol) were added thereto. The reaction solution was stirred at 100° C. overnight. Upon completion of the reaction, 100 mL of water was added thereto, followed by extraction three times with ethylacetate. An organic layer was dried with anhydrous sodium sulfate and filtered under reduced pressure. After the filtered organic layer was concentrated under reduced pressure, residues obtained therefrom were purified by MPLC (dichloromethane:methanol=20:1(v/v)) to obtain 6.8 g of the title compound (yield: 52%).

¹H-NMR (300 MHz, CDCl₁₃): δ 8.92-8.91 (m, 1H), 8.20-8.16 (m, 1H), 7.92 (s, 1H), 7.86-7.83 (m, 2H), 7.69-7.56 (m, 4H), 7.34 (s, 1H), 3.83 (s, 3H).

[Process-4] Preparation of 3-(1-methyl-1H-imidazol-4-yl)-1-(4-(trifluoromethyl)phenyl)-1H-indol-5-amine

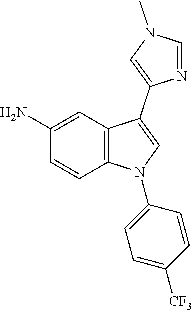 [SMILES: Cn1cnc(-c2cn(-c3ccc(C(F)(F)F)cc3)c3ccc(N)cc23)c1]

3-(1-methyl-1H-imidazol-4-yl)-5-nitro-1-(4-(trifluoromethyl)phenyl)-1H-indole (6.8 g, 17.60 mmol) obtained in [Process-3] was dissolved in 140 mL of ethanol and Pd/C (1.9 g, 0.2 wt %) was added thereto. The reaction solution was stirred under a hydrogen gas for 2 hours. Upon completion of the reaction, the reaction solution was filtered through CELITE and washed with methanol. After the filtered organic layer was concentrated under reduced pressure, residues obtained therefrom were purified by MPLC (dichloromethane:methanol=20:1(v/v)) to obtain 4.5 g of the title compound (yield: 72%).

¹H-NMR (300 MHz, CDCl₃): δ 7.83-7.62 (m, 5H), 7.58-7.43 (m, 2H), 7.23-7.18 (m, 3H), 6.75-6.67 (m, 1H), 3.77 (s, 3H).

[Process-5] Preparation of N-(3-(1-methyl-1H-imidazol-4-yl)-1-(4-(trifluoromethyl phenyl)-1H-indol-5-yl) acrylamide

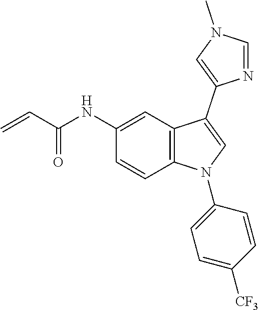 [SMILES: C=CC(=O)Nc1ccc2c(c1)c(-c1cn(C)cn1)cn2-c1ccc(C(F)(F)F)cc1]

3-(1-methyl-1H-imidazol-4-yl)-1-(4-(trifluoromethyl)phenyl)-1H-indol-5-amine (4.5 g, 12.62 mmol) obtained in [Process-4] was dissolved in 60 mL of a tetrahydrofuran:water mixture (3:1, v/v), and sodium bicarbonate (1.6 g, 18.94 mmol) was added thereto. The reaction solution was cooled to a temperature of 0 to 5° C. and stirred while slowing adding acryloyl chloride (1.2 mL, 13.25 mmol) dropwise thereto. Upon completion of the reaction, 40 mL of water was added thereto, followed by extraction three times with 40 mL of ethylacetate. An organic layer was dried with anhydrous sodium sulfate and filtered under reduced pressure. After the filtered organic layer was concentrated under reduced pressure, residues obtained therefrom were purified by MPLC (dichloromethane:methanol=30:1(v/v)) to obtain 3.8 g of the title compound (yield: 74%).

¹H-NMR (300 MHz, DMSO-d₆): δ 10.19 (s, 1H), 8.50 (s, 1H), 7.96-7.88 (m, 5H), 7.69-7.59 (m, 3H), 7.42 (s, 1H), 6.54-6.45 (m, 1H), 6.30-6.23 (m, 1H), 5.76-5.72 (m, 1H), 3.74 (s, 3H).

MS (ESI⁺, m/z): 411.2 [M+H]⁺

### Example 2: N-(3-(1-cyclopropyl-1H-imidazol-4-yl)-1-(4-(trifluoromethyl)phenyl)-1H-indol-5-yl) acrylamide

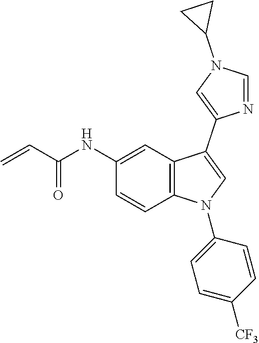 [SMILES: C=CC(=O)Nc1ccc2c(c1)c(-c1cn(C3CC3)cn1)cn2-c1ccc(C(F)(F)F)cc1]

20 mg of the title compound (yield: 4%) was obtained by repeating the processes of Example 1, except that tributyl-(1-cyclopropylimidazol-4-yl)stannane (621 mg, 1.56 mmol) was used in [Process-3] of Example 1 instead of tributyl-(1-methylimidazol-4-yl)stannane.

¹H-NMR (300 MHz, CDCl₃): δ 8.37 (s, 1H), 7.81-7.76 (m, 3H), 7.67-7.55 (m, 4H), 7.49-7.34 (m, 3H), 6.50-6.44 (m, 1H), 6.34-6.31 (m, 1H), 5.80 (d, J=10.0 Hz, 1H), 3.43-3.40 (m, 1H), 1.12-1.01 (m, 4H).

MS (ESI⁺, m/z): 437.4 [M+H]⁺

### Example 3: N-(3-(furan-2-yl)-1-(4-(trifluoromethyl)phenyl)-1H- indol-5-yl)acrylamide

[Process-1] Preparation of 3-(furan-2-yl)-5-nitro-1-(4-(trifluoromethyl)phenyl)-1H- indole

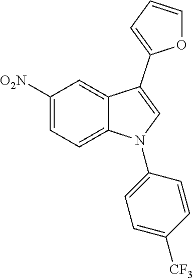 [SMILES: O=[N+]([O-])c1ccc2c(c1)c(-c1ccco1)cn2-c1ccc(C(F)(F)F)cc1]

3-bromo-5-nitro-1-(4-(trifluoromethyl)phenyl)-1H-indole (300 mg, 0.77 mmol) obtained in [Process-2] of Example 1 was dissolved in 5 mL of a 1,4-dioxane:water mixture (4:1, v/v), and 2-(furan-2-yl)-4,4,5,5-tetramethyl-1,3,2-dioxaborolan (211 mg, 1.09 mmol), [1,1′-bis(diphenylphosphino)bisferrocenyl]palladium dichloride ([Pd(dppf)Cl₂]) (60 mg, 0.08 mmol), and sodium carbonate (165 mg, 1.55 mmol) were added thereto, respectively. The reaction solution was stirred at 100° C. for 2 hours. Upon completion of the reaction, the reaction solution was cooled to room temperature, and 20 mL of water was added thereto, followed by extraction three times with 20 mL of ethylacetate. An organic layer was dried with anhydrous sodium sulfate and filtered under reduced pressure, and the filtered organic layer was concentrated under reduced pressure. Residues obtained therefrom were purified by MPLC (ethylacetate:hexane=1:2(v/v)) to obtain 100 mg of the title compound (yield: 62%).

¹H-NMR (300 MHz, DMSO-d₆): δ 8.92 (s, 1H), 8.43 (s, 1H), 8.20-8.14 (m, 1H), 8.04-7.95 (m, 4H), 7.86-7.77 (m, 2H), 6.95 (d, J=2.9 Hz, 1H), 6.68-6.66 (m, 1H).

### [Process-2] Preparation of N-(3-(furan-2-yl)-1-(4-(trifluoromethyl)phenyl)-1H-indol-5-yl)acrylamide

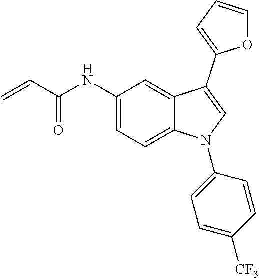 [SMILES: C=CC(=O)Nc1ccc2c(c1)c(-c1ccco1)cn2-c1ccc(C(F)(F)F)cc1]

44 mg of the title compound (yield: 21%) was obtained by repeating the processes of Example 1, except that 3-(2-furyl)-5-nitro-1-(4-(trifluoromethyl)phenyl)indole (180 mg, 0.49 mmol) obtained in [Process-1] was used in [Process-4] of Example 1 instead of 3-(1-methyl-1H-imidazol-4-yl)-5-nitro-1-(4-(trifluoromethyl)phenyl)-1H-indole.

¹H-NMR (300 MHz, DMSO-d₆): δ 10.2 (s, 1H), 8.50 (s, 1H), 8.13 (s, 1H), 7.97-7.90 (m, 4H), 7.76-7.74 (m, 1H), 7.71-7.69 (m, 1H), 7.59-7.56 (m, 1H), 6.72-6.71 (m, 1H), 6.66-6.65 (m, 1H), 6.64-6.47 (m, 1H), 6.31-6.30 (m, 1H), 5.78-5.74 (m, 1H).

MS (ESI⁺, m/z): 397.1 [M+H]⁺

### Example 4: N-(3-(1-cyclobutyl-1H-imidazol-4-yl)-1-(4-(trifluoromethyl)phenyl)-1H-indol-5-yl)acrylamide

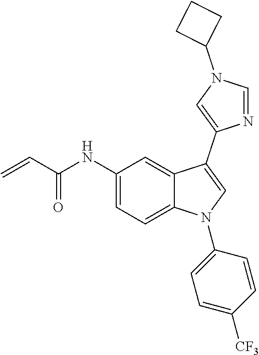 [SMILES: C=CC(=O)Nc1ccc2c(c1)c(-c1cn(C3CCC3)cn1)cn2-c1ccc(C(F)(F)F)cc1]

17 mg of the title compound (yield: 10%) was obtained by repeating the processes of Example 1, except that tributyl-(1-cyclobutylimidazol-4-yl)stannane (1.1 g, 2.59 mmol) was used in [Process-3] of Example 1 instead of tributyl-(1-methylimidazol-4-yl)stannane.

¹H-NMR (300 MHz, CDCl₃): δ 8.34 (s, 1H), 7.81-7.67 (m, 3H), 7.61-7.55 (m, 2H), 7.45-7.35 (m, 5H), 6.49-6.25 (m, 2H), 5.80 (d, J=10.0 Hz, 1H), 4.72-4.61 (m, 1H), 2.62-2.52 (m, 2H), 2.50-2.39 (m, 2H), 1.99-1.88 (m, 2H).

MS (ESI⁺, m/z): 451.5 [M+H]⁺

### Example 5: N-(3-(2-fluorophenyl)-1-(4-(trifluoromethyl)phenyl)-1H-indol-5-yl)acrylamide

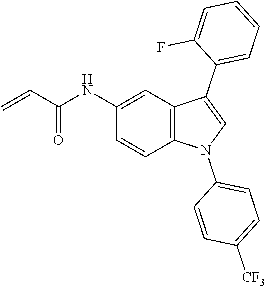 [SMILES: C=CC(=O)Nc1ccc2c(c1)c(-c1ccccc1F)cn2-c1ccc(C(F)(F)F)cc1]

20 mg of the title compound (yield: 24%) was used by repeating the processes of Example 3, except that (2-fluorophenyl)borate (27 mg, 0.13 mmol) was used in Example 3 instead of 2-(furan-2-yl)-4,4,5,5-tetramethyl-1,3,2-dioxaborolan.

¹H-NMR (300 MHz, DMSO-d₆): δ 10.19 (s, 1H), 8.21 (s, 1H), 8.01 (s, 1H), 7.98-7.91 (m, 4H), 7.75-7.73 (m, 2H), 7.59-7.56 (m, 1H), 7.44-7.33 (m, 3H), 6.50-6.41 (m, 1H), 6.27-6.22 (m, 1H), 5.75-5.71 (m, 1H).

MS (ESI⁺, m/z): 425.1 [M+H]⁺

### Example 6:2-fluoro-N-(3-(1-methyl-1H-imidazol-4-yl)-1-(4-(trifluoromethyl)phenyl)-1H-indol-5-yl) acrylamide

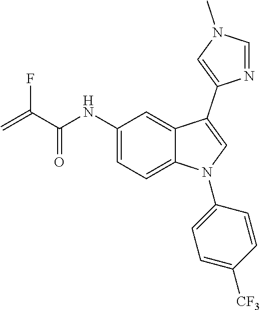 [SMILES: C=C(F)C(=O)Nc1ccc2c(c1)c(-c1cn(C)cn1)cn2-c1ccc(C(F)(F)F)cc1]

After dissolving 3-(1-methyl-1H-imidazol-4-yl)-1-(4-(trifluoromethyl)phenyl)-1H-indol-5-amine (55 mg, 0.15 mmol) obtained in [Process-4] of Example 1 in 1.1 mL of tetrahydrofuran, and 2-fluoroacrylate (15 mg, 0.17 mmol), diisopropylethylamine (0.1 mL, 18.94 mmol), and 1-[bis(dimethylamino)methylene]-1H-1,2,3-triazolo[4,5-b]pyridinium 3-oxide hexafluorophosphate (HATU, 88 mg, 0.23 mmol) were added thereto at 0° C. The reaction solution was heated to room temperature and stirred overnight. Upon completion of the reaction, 5 mL of water was added thereto, followed by extraction three times with ethylacetate. An organic layer was dried with anhydrous sodium sulfate and filtered under reduced pressure. After the filtered organic layer was concentrated under reduced pressure, residues obtained therefrom were purified by MPLC (dichloromethane:methanol=15:1(v/v)) to obtain 10 mg of the title compound (yield: 15%).

¹H-NMR (300 MHz, DMSO-d₆): δ 10.33 (s, 1H), 8.49 (s, 1H), 7.98 (s, 1H), 7.95-7.87 (m, 4H), 7.69-7.61 (m, 3H), 7.45 (s, 1H), 5.81-5.64 (m, 1H), 5.45-5.38 (m, 1H), 3.74 (s, 3H).

MS (ESI⁺, m/z): 429.2 [M+H]⁺

### Example 7: N-(3-(1-(2-methoxyethyl)-1H-imidazol-4-yl)-1-(4-(trifluoromethyl)phenyl)-1H-indol-5-yl)acrylamide

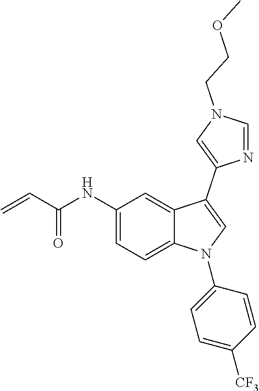 [SMILES: C=CC(=O)Nc1ccc2c(c1)c(-c1cn(CCOC)cn1)cn2-c1ccc(C(F)(F)F)cc1]

10 mg of the title compound (yield: 30%) was obtained by repeating the processes of Example 1, except that tributyl-[1-(2-methoxyethyl)imidazol-4-yl) stannane (43 mg, 0.10 mmol) was used in [Process-3] of Example 1 instead of tributyl-(1-methylimidazol-4-yl)stannane.

¹H-NMR (300 MHz, DMSO-d₆): δ 10.19 (s, 1H), 8.50 (s, 1H), 7.97 (s, 1H), 7.94-7.87 (m, 4H), 7.72 (s, 1H), 7.68-7.66 (m, 2H), 7.48 (s, 1H), 6.54-6.48 (m, 1H), 6.29-6.23 (m, 1H), 5.76-5.72 (m, 1H), 4.23 (t, J=5.1 Hz, 2H), 3.68 (t, J=5.1 Hz, 2H), 3.29 (s, 3H).

MS (ESI⁺, m/z): 455.2 [M+H]⁺

### Example 8: N-(3-(1-cyclopropyl-1H-imidazol-4-yl)-1-(4-(trifluoromethyl) phenyl)-1H-pyrrolo[2,3-b]pyridin-5-yl)acrylamide

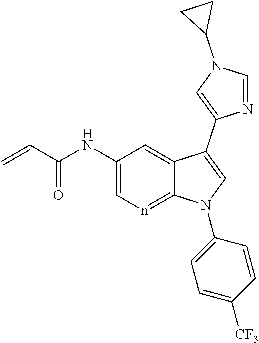

7 mg of the title compound (yield: 10%) was obtained by repeating the processes of Example 1, except that 5-bromo-1H-pyrrolo[2,3-b]pyridine (1.0 g, 5.07 mmol) was used in [Process-1] of Example 1 instead of 5-nitro-1H-indole and tributyl-(1-cyclopropylimidazol-4-yl)stannane (411 mg, 1.03 mmol) was used in [Process-3] instead of tributyl-(1-methylimidazol-4-yl)stannane.

¹H-NMR (300 MHz, DMSO-d₆): δ 10.41 (s, 1H), 8.94 (d, J=2.3 Hz, 1H), 8.66 (d, J=2.3 Hz, 1H), 8.34-8.30 (m, 3H), 7.93-7.90 (m, 2H), 7.83 (d, J=1.2 Hz, 1H), 7.60 (d, J=1.2 Hz, 1H), 6.51-6.45 (m, 1H), 6.34-6.28 (m, 1H), 5.82-5.75 (m, 2H), 1.12-1.01 (m, 4H).

MS (ESI⁺, m/z): 438.4 [M+H]⁺

### Example 9: N-(3-(1-methyl-1H-imidazol-4-yl)-1-(4-(trifluoromethyl)phenyl)-1pyrrolo[2,3-b]pyridin-5-yl)acrylamide

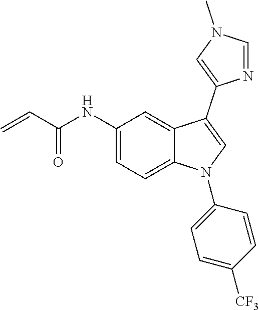 [SMILES: C=CC(=O)Nc1ccc2c(c1)c(-c1cn(C)cn1)cn2-c1ccc(C(F)(F)F)cc1]

30 mg of the title compound (yield: 26%) was obtained by repeating the processes, except that tributyl-(1-methylimidazol-4-yl)stannane (576 mg, 1.55 mmol) was used in Example 8 instead of tributyl-(1-cyclopropylimidazol-4-yl)stannane.

¹H-NMR (300 MHz, CDCl₃): δ 8.88 (brs, 1H), 8.29 (d, J=2.4 Hz, 1H), 8.10-8.00 (m, 3H), 7.77-7.75 (m, 2H), 7.55-7.51 (m, 2H), 7.27-7.26 (m, 1H), 6.54-6.48 (m, 1H), 6.37-6.28 (m, 1H), 5.85 (d, J=10.8 Hz, 1H), 3.77 (s, 3H).

MS (ESI⁺, m/z): 412.4 [M+H]⁺

### Example 10: N-(6-chloro-3-(1-methyl-1H-imidazol-4-yl)-1-(4-(trifluoromethyl)phenyl)-1H-indol-5-yl)acrylamide

[Process-1] Preparation of 5-bromo-6-chloro-1-(4-(trifluoromethyl)phenyl)-1H-indole

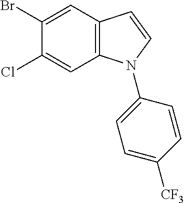 [SMILES: FC(F)(F)c1ccc(-n2ccc3cc(Br)c(Cl)cc32)cc1]

5-bromo-6-chloro-1H-indole (5.0 g, 21.69 mmol) was dissolved in 50 mL of dimethyl sulfoxide, and 4-bromobenzotrifluoride (7.4 g, 32.54 mmol), potassium carbonate (6.0 g, 43.39 mmol), and N,N′-dimethylethylenediamine (0.2 g, 2.17 mmol) were added thereto and then copper (I) iodide (4.9 g, 26.03 mmol) was added thereto. The reaction solution was stirred at 110° C. overnight. Upon completion of the reaction, the reaction solution was cooled to room temperature and 100 mL of water was added thereto, followed by extraction three times with ethylacetate. An organic layer was dried with anhydrous sodium sulfate and filtered under reduced pressure. After the filtered organic layer was concentrated under reduced pressure, residues obtained therefrom were purified by MPLC (ethylacetate: hexane=1:20(v/v)) to obtain 5.34 g of the title compound (yield: 66%).

¹H-NMR (300 MHz, CDCl₃): 8 7.94 (s, 1H), 7.82 (d, J=8.5 Hz, 2H), 7.66 (s, 1H), 7.59 (d, J=8.3 Hz, 2H), 7.35 (d, J=3.3 Hz, 1H), 6.65 (d, J=3.0 Hz, 1H).

[Process-2] Preparation of t-butyl(6-chloro-1-(4-(trifluoromethyl)phenyl)-1H- indol-5-yl)carbamate

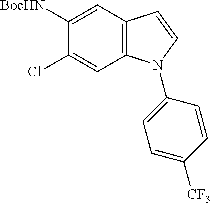 [SMILES: CC(C)(C)OC(=O)Nc1cc2ccn(-c3ccc(C(F)(F)F)cc3)c2cc1Cl]

5-bromo-6-chloro-1-(4-(trifluoromethyl)phenyl)-1H-indole (5.3 g, 14.25 mmol) obtained in [Process-1] was dissolved in 80 mL of toluene, and tris(dibenzylideneacetone)dipalladium ([Pd₂(dba)₃])(1.3 g, 1.43 mmol), sodium t-butoxide (2.7 g, 28.49 mmol), t-butyl carbamate (2.0 g, 17.09 mmol), and (2-biphenyl)di-t-butylphosphine (JohnPhos, 0.4 g, 1.43 mmol) were added thereto. The reaction solution was stirred at 90° C. for 4 hours. Upon completion of the reaction, the reaction solution was cooled to room temperature and 100 mL of water was added thereto, followed by extraction three times with ethylacetate. An organic layer was dried with anhydrous sodium sulfate and filtered under reduced pressure. After the filtered organic layer was concentrated under reduced pressure, residues obtained therefrom were purified by MPLC (ethylacetate: hexane=1:20(v/v)) to obtain 2.7 g of the title compound (yield: 46%).

¹H-NMR (300 MHz, CDCl₃): δ 8.39 (s, 1H), 7.79 (d, J=8.3 Hz, 2H), 7.60-7.58 (m, 3H), 7.32 (d, J=3.3 Hz, 1H), 6.96 (brs, 1H), 6.67-6.65 (m, 1H), 1.56 (s, 9H).

[Process-3] Preparation of 6-chloro-1-(4-(trifluoromethyl)phenyl)-1H-indol-5-amine hydrochloride

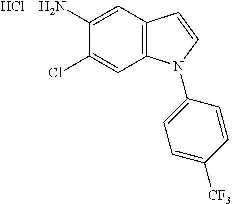 [SMILES: Cl ; Nc1cc2ccn(-c3ccc(C(F)(F)F)cc3)c2cc1Cl]

A 4 N hydrochloric acid dioxane solution (46.1 mL, 184.30 mmol) was slowly added dropwise to t-butyl (6-chloro-1-(4-(trifluoromethyl) phenyl)-1H-indol-5-yl)carbamate (2.5 g, 6.14 mmol) obtained in [Process-2] at 0° C. The reaction solution was stirred at 0° C. for 4 hours. Upon completion of the reaction, a formed solid was collected by filtration, washed several times with hexane, and dried to obtain 1.96 g of the target compound (yield: 92%).

¹H-NMR (300 MHz, DMSO-d₆): δ 7.94 (d, J=8.5 Hz, 2H), 7.84 (d, J=8.6 Hz, 2H), 7.79 (d, J=3.5 Hz, 1H), 7.72 (s, 1H), 7.41 (s, 1H), 6.72 (d, J=3.2 Hz, 1H), 3.85 (brs, 3H).

[Process-4] Preparation of N-(6-chloro-1-(4-(trifluoromethyl)phenyl)-1H-indol-5-yl)acrylamide

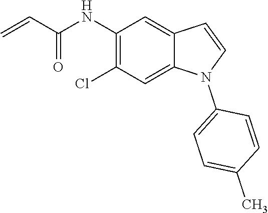 [SMILES: C=CC(=O)Nc1cc2ccn(-c3ccc(C)cc3)c2cc1Cl]

6-chloro-1-(4-(trifluoromethyl)phenyl)-1H-indol-5-amine hydrochloride (0.6 g, 1.73 mmol) obtained in [Process-3] was dissolved in 12 mL of tetrahydrofuran, and tri-ethylamine (0.9 mL, 6.91 mmol) was added dropwise thereto. The reaction solution was cooled to a temperature of 0 to 5° C., and acryloyl chloride (0.2 mL, 2.25 mmol) was slowly added dropwise thereto, followed by stirring at room temperature for 2.5 hours. Upon completion of the reaction, 25 mL of water was added thereto, followed by extraction three times with ethylacetate. An organic layer was dried with anhydrous sodium sulfate and filtered under reduced pressure. After the filtered organic layer was concentrated under reduced pressure, residues obtained therefrom were purified by MPLC (ethylacetate:hexane=1:5(v/v)) to obtain 0.5 g of the title compound (yield: 79%).

¹H-NMR (300 MHz, CDCl₃): δ 8.76 (brs, 1H), 7.82-7.76 (m, 3H), 7.61-7.59 (m, 3H), 7.35 (d, J=3.3 Hz, 1H), 6.72-6.71 (m, 1H), 6.51-6.29 (m, 2H), 5.82 (dd, J=1.4, 10.0 Hz, 1H).

[Process-5] Preparation of N-(3-bromo-6-chloro-1-(4-(trifluoromethyl)phenyl)-1H-indol-5-yl)acrylamide

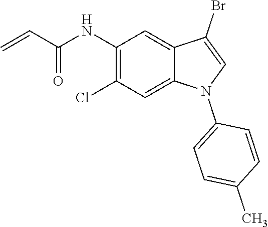 [SMILES: C=CC(=O)Nc1cc2c(Br)cn(-c3ccc(C)cc3)c2cc1Cl]

0.4 g of the title compound (yield: 66%) was obtained by repeating the processes of Example 1, except that N-(6-chloro-1-(4-(trifluoromethyl)phenyl)-1H-indol-5-yl)acrylamide (0.5 g, 1.34 mmol) was used in [Process-2] of Example 1 instead of 5-nitro-1-(4-(trifluoromethyl)phenyl)-1H-indole and the reaction solution was stirred at 0° C. for 2 hours.

¹H-NMR (300 MHz, CDCl₃): δ 8.72 (brs, 1H), 7.82 (d, J=8.4 Hz, 2H), 7.77 (s, 1H), 7.59-7.56 (m, 3H), 7.40 (s, 1H), 6.54-6.48 (m, 1H), 6.39-6.30 (m, 1H), 5.84 (dd, J=1.2, 10.1 Hz, 1H).

[Process-6] Preparation of N-(6-chloro-3-(1-methyl-1H- imidazol-4-yl)-1-(4-(trifluoromethyl)phenyl)-1H-indol-5-yl) acrylamide

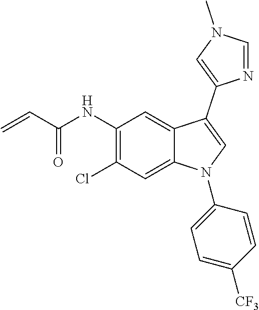 [SMILES: C=CC(=O)Nc1cc2c(-c3cn(C)cn3)cn(-c3ccc(C(F)(F)F)cc3)c2cc1Cl]

9 mg of the title compound (yield: 2%) was obtained by repeating the processes of Example 1, except that N-(3-bromo-6-chloro-1-(4-(trifluoromethyl)phenyl)-1H- indol-5-yl)acrylamide (0.4 g, 0.88 mmol) was used in [Process-3] of Example 1 instead of 3-bromo-5-nitro-1-(4-(trifluoromethyl)phenyl)-1H-indole and the reaction solution was stirred at 100° C. for 2 hours.

¹H-NMR (300 MHz, DMSO-d₆): δ 9.81 (s, 1H), 8.41 (s, 1H), 8.05 (s, 1H), 7.98-7.90 (m, 4H), 7.80 (s, 1H), 7.69 (s, 1H), 7.55 (s, 1H), 6.66-6.56 (m, 1H), 6.26 (dd, J=2.0, 17.0 Hz, 1H), 5.80 (d, J=10.0 Hz, 1H), 3.74 (s, 3H).

MS (ESI⁺, m/z): 445.1 [M+H]⁺

### Experimental Example 1: TEAD Reporter Activity Inhibition Test

The ability of the synthetic compounds to inhibit transcription of a target gene by TEAD was measured. This evaluation method was a method of measuring transcriptional activity of TEAD by measuring luminescence intensity of luciferase expressed in the case where TEAD binds to a target gene and activates transcription thereof, by using MCF7 cell line (BPS Bioscienc, Inc., USA) constructed by introducing firefly luciferase reporter gene into GTIIC (5′-ACATTCCA-3′), which is a specific binding structure of TEAD. The cell line was cultured in a MEM medium supplemented with 10% FBS, 1% Penicillin/Streptomycin, 1% non-essential amino acids, 10 μg/ml insulin, and 400 μg/ml Geneticin. In the test of inhibiting the TEAD reporter activity, Geneticin was excluded. The cells were aliquoted into a white 96-well plate at a density of 4×10⁴ cells/100 μl and cultured for 6 hours. 50 μl of the test compound diluted to 3× concentration was mixed with each well. After culturing the cell line for 24 hours, signals of luciferase were measured by luminescence according to manufacturer's protocols by using ONE-Glo luciferase assay system (Promega, E6120). A 50% inhibitory concentration (IC₅₀) on TEAD transcription activity was calculated by using GraphPad Prism 9.

An IC₅₀ value less than 100 nM was expressed as A, an IC₅₀ value not less than 100 nM but less than 500 nM was expressed as B, and an IC₅₀ value not less than 500 nM was expressed as C.

TABLE 1

| Synthetic compound | IC₅₀, nM |
|---|---|
| 1 | A |
| 2 | A |
| 3 | A |
| 4 | A |
| 5 | A |
| 6 | B |
| 7 | A |
| 8 | A |
| 9 | A |
| 10 | B |

### Experimental Example 2: Cell Growth Inhibition Test

The ability of the synthetic compounds to inhibit the growth of NCI-H226 cells was identified. NCI-H226, which is an NF2 gene-deficient mesothelioma cancer cell line, was cultured in a RPMI 1640 medium supplemented with 10% FBS and 1% Penicillin/Streptomycin. The cultured cells were aliquoted into a 96-well plate at a density of 0.7×10³ cells/100 μl and cultured for 24 hours, and then 100 μl of a test compound diluted to 2× concentration was mixed with each well and cultured for 6 days. SRB test method was used to measure cell growth inhibition, and a 50% inhibitory concentration (GI₅₀) on cell growth by the compound was calculated by using GraphPad Prism 9.

A GI₅₀value less than 100 nM was expressed as A and a GI₅₀ value not less than 100 nM was expressed as B.

TABLE 2

| Synthetic compound | GI₅₀, nM |
|---|---|
| 1 | A |
| 2 | A |
| 3 | A |
| 4 | A |
| 5 | A |
| 7 | A |
| 8 | A |
| 9 | A |

While the present disclosure has been particularly shown and described with reference to exemplary embodiments thereof. It will be understood by those of ordinary skill in the art that various changes in form and details may be made therein without departing from the spirit and scope of the present disclosure as defined by the following claims. The preferred embodiments should be considered in descriptive sense only and not for purposes of limitation. Therefore, the scope of the present disclosure is defined not by the detailed description but by the appended claims, and all differences within the scope will be construed as being included in the present disclosure.

1. A compound selected from a compound of Formula 1 below, enantiomers, diastereomers, solvates and hydrates thereof, and pharmaceutically acceptable salts thereof: 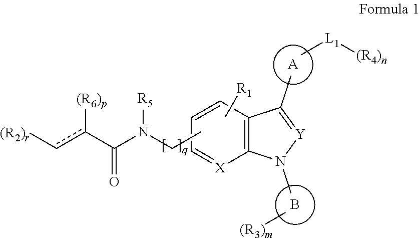
wherein in Formula 1,
R₁ is hydrogen, halogen, C₁-₆ alkyl, halo C₁-₆ alkyl, C₁-₆ alkoxy, or cyano;
R₂ is each independently hydrogen, halogen, C₁-₆ alkyl, halo C₁-₆ alkyl, or substituted or unsubstituted —(CH₂)ₐ—N(Q₁)(Q₂),
wherein Q₁ and Q₂ are each independently hydrogen or C₁-₄ alkyl;
\textcircled{A} is carbocyclyl or heterocyclyl;
\textcircled{B} is C₆-₁₀ aryl or C₄-₁₀ heteroaryl;
L₁ is absent, a bond, C₁-₃ alkyl, or halo C₁-₃ alkyl;
R₃ and R₄ are each independently hydrogen, halogen, cyano, amino, C₁-₆ alkyl, halo C₁-₆ alkyl, C₁-₆ alkoxy, halo C₁-₆ alkoxy, C₁-₆ alkoxyalkyl, substituted or unsubstituted C₃-₆ carbocyclyl, substituted or unsubstituted C₆-₁₀ aryl, substituted or unsubstituted C₂-₆ heterocyclyl, or substituted or unsubstituted C₄-₁₀ heteroaryl;
R₅ is hydrogen, C₁-₆ alkyl, or halo C₁-₆ alkyl;
R₆ is each independently hydrogen, halogen, C₁-₆ alkyl, or halo C₁-₆ alkyl;
X and Y are each independently —C— or —N—; and
a, m, n, q, r, and p are each independently an integer from 0 to 3.

2. The compound of claim 1, wherein \textcircled{A} is C₃-₆ cycloalkyl, C₆-₁₀ aryl, C₂-₆ heterocycloalkyl, C₁-₁₀ heteroaryl, or C₆-₁₄ fused heteroaryl,
wherein the C₂-₆ heterocycloalkyl, the C₁-₁₀ heteroaryl, or the C₆-₁₄ fused heteroaryl each independently comprise 1 to 4 hetero atoms selected from N, O, and S.

3. The compound of claim 1, wherein \textcircled{A} is a phenyl group, a pyridinyl group, a pyrazinyl group, a pyrazolyl group, an imidazolyl group, a thiophenyl group, a furanyl group, or an oxazole group.

4. The compound of claim 1, wherein \textcircled{B} is a phenyl group or a pyridinyl group.

5. The compound of claim 1, wherein R₁ is hydrogen, halogen, or cyano.

6. The compound of claim 1, wherein R₂ is each independently hydrogen or halogen.

7. The compound of claim 1, wherein R₅ is hydrogen.

8. The compound of claim 1, wherein R₆ is each independently hydrogen or halogen.

9. The compound of claim 1, wherein L₁ is a bond; and
R₃ and R₄ are each independently hydrogen, halogen, cyano, C₁-₆ alkyl, halo C₁-₆ alkyl, C₁-₆ alkoxy, C₁-₆ alkoxyalkyl, substituted or unsubstituted C₃-₆ cycloalkyl, substituted or unsubstituted C₆-₁₀ aryl, or substituted or unsubstituted C₂-₆ heterocycloalkyl.

10. The compound of claim 1, wherein
\textcircled{A} is a phenyl group, an imidazolyl group, or a furanyl group;
\textcircled{B} is a phenyl group;
L₁ is a bond;
R₃ is halo C₁-₆ alkyl;
R₄ is hydrogen, halogen, C₁-₆ alkyl, halo C₁-₆ alkyl, C₁-₆ alkoxy, C₁-₆ alkoxyalkyl, or C₃-₆ cycloalkyl;
R₁ is hydrogen or halogen;
R₂ and R5 are each hydrogen;
R₆ is each independently hydrogen or halogen;
X and Y are each independently —C— or —N—; and
m, n, q, r, and p are each independently an integer from 0 to 2.

11. The compound of claim 1, wherein the compound is selected from compounds shown below, enantiomers, diastereomers, solvates and hydrates thereof, and pharmaceutically acceptable salts thereof:
1. N-(3-(1-methyl-1H-imidazol-4-yl)-1-(4-(trifluoromethyl)phenyl)-1H-indol-5-yl)acrylamide;
2. N-(3-(1-cyclopropyl-1H-imidazol-4-yl)-1-(4-(trifluoromethyl)phenyl)-1H-indol-5-yl)acrylamide;
3. N-(3-(furan-2-yl)-1-(4-(trifluoromethyl)phenyl)-1H-indol-5-yl)acrylamide;
4. N-(3-(1-cyclobutyl-1H-imidazol-4-yl)-1-(4-(trifluoromethyl)phenyl)-1H-indol-5-yl)acrylamide;
5. N-(3-(2-fluorophenyl)-1-(4-(trifluoromethyl)phenyl)-1H-indol-5-yl)acrylamide;
6. 2-fluoro-N-(3-(1-methyl-1H-imidazol-4-yl)-1-(4-(trifluoromethyl)phenyl)-1H-indol-5-yl)acrylamide;
7. N-(3-(1-(2-methoxyethyl)-1H-imidazol-4-yl)-1-(4-(trifluoromethyl)phenyl)-1H-indol-5-yl)acrylamide;
8. N-(3-(1-cyclopropyl-1H-imidazol-4-yl)-1-(4-(trifluoromethyl)phenyl)-1H-pyrrolo[2,3-b]pyridin-5-yl)acrylamide;
9. N-(3-(1-methyl-1H-imidazol-4-yl)-1-(4-(trifluoromethyl)phenyl)-1H-pyrrolo[2,3-b]pyridin-5-yl)acrylamide; and
10. N-(6-chloro-3-(1-methyl-1H-imidazol-4-yl)-1-(4-(trifluoromethyl)phenyl)-1H-indol-5-yl)acrylamide.

12. A method for treating or preventing a disease caused by transcriptional enhancer associate domain (TEAD) activation in a subject, the method comprising administering to the subject a pharmaceutical composition comprising, as an active ingredient, the compound of claim 1.

13. The method of claim 12, wherein the composition inhibits Yes associated protein (YAP)-transcriptional enhancer associate domain (TEAD) binding.

14. The method of claim 12, wherein the disease is a cancer or tumor.

15. A pharmaceutical composition comprising a compound of claim 1 and a pharmaceutically acceptable additive.
